(19) United States
(12) Patent Application Publication
(10) Pub. No.: US 2024/0341711 A1
(43) Pub. Date: Oct. 17, 2024

(54) System and Method for Monitoring Lesion Progression Over Multiple Medical Scans

(71) Applicant: Wisconsin Alumni Research Foundation, Madison, WI (US)

(72) Inventors: Robert Jeraj, Madison, WI (US); Victor Fernandes, Madison, WI (US)

(21) Appl. No.: 18/298,468

(22) Filed: Apr. 11, 2023

(51) Int. Cl. A61B 6/00; G06T 7/00

(52) U.S. Cl. CPC A61B 6/5217; G06T 7/0012; G06T 2207/30096

(57) ABSTRACT

A method and apparatus for tracking disease progression as revealed by multiple lesions matches lesions between time-separated scans of a patient through a global process which assesses overlap between all pairs in the scan sequence and links lesion in different images to maximize overlap globally.

### STATEMENT REGARDING FEDERALLY SPONSORED RESEARCH OR DEVELOPMENT

--

### CROSS REFERENCE TO RELATED APPLICATION

--

### BACKGROUND OF THE INVENTION

The present invention relates to techniques for assessing disease treatment and, in particular, to a computerized system linking lesions among different medical scans taken over time to better reveal disease progression.

Metastasis is the leading cause of cancer-related mortality. In metastasis, cells of a primary cancer break away from where they were first formed and travel through the body to create new lesions. Each metastatic lesion may respond differently to treatment and, accordingly, lesion-level assessment may be necessary for a complete understanding of disease response. Such lesion-level assessment, however, is difficult as it requires manual matching of as many as hundreds of corresponding lesions, which is a tedious, subjective, and error-prone task, and is therefore rarely performed in practice.

U.S. Pat. No. 10,445,878 entitled “Image Enhancement System for Bone Disease Evaluation,” assigned to the assignees of the present invention and hereby incorporated by reference, describes a lesion monitoring system for tumors in the skeletal system, the latter of which presents an articulated but rigid target simplifying the registration of longitudinally acquired images.

US patent application 2022/0338805 entitled “System and Method for Monitoring Multiple Lesions,” assigned to the assignees of the present invention and hereby incorporated by reference, describes a lesion monitoring system monitoring metastatic lesions distributed over the entire patient anatomy and identifying lesions that split or combine in successive pairs of scans.

### SUMMARY OF THE INVENTION

The present inventors have determined that a pair-wise matching of lesions in sequential images can lead to substantial errors in assessing the disease progression, for example, when a lesion disappears, or falls below the detectability limit, in one image and reappears and is improperly interpreted as a remission plus a new lesion. Lesion mismatching in an early pair of images can propagate through successive matching operations. The present invention addresses this problem by using a global assessment of lesion overlap to link lesions among different images. Although this evaluation of overlap in non-sequential images might be expected to introduce errors in lesion identification as the images become mis-registered over time, preliminary experiments using a global assessment indicate a high accuracy of over 90% in accurately tracking lesions between scans

Specifically then, in one embodiment, the invention provides an apparatus for assessing treatment using an electronic computer receiving a set of at least three scans of tissue of the patient revealing diseased tissue. A program executing on the computer determines lesion volumes in the scan as assigned to identifiers and an overlapping of lesion volumes between all pairs of scans of the set to provide a set of overlap measures for each pair of scans for each pair of identifiers. The lesions identifiers in different scans are linked in a way that globally maximizes the overlap measures of the set over all of the scans. This result is used to output a display indicating a lesion change identified to given linked lesions.

It is thus a feature of at least one embodiment of the invention to better link lesions in different images taken over time so as to provide a more accurate characterization of the effectiveness of the treatment and shrinking or eliminating those lesions.

The program may further identify a set of tissue structures, for example, organs, and link the output of lesion change to a tissue structure.

It is thus a feature of at least one embodiment of the invention to allow the clinician to focus on lesion changes in particular tissue structures to provide a more nuanced understanding of disease progression.

The output may be a graphic display indicating a linkage between lesions of different scans superimposed on at least one scan image.

It is thus a feature of at least one embodiment of the invention to provide anatomical context to the progression of the lesions over time.

The program may further receive input from a user to alter the linking of the lesion and to change the output according to that input.

It is thus a feature of at least one embodiment of the invention to provide an output that reflects additional input from a trained user.

The display may further output an uncertainty value on the graphics display associated with the linkage.

It is thus a feature of at least one embodiment of the invention to guide the clinician to uncertain linkages that may warrant additional inspection.

The lesion volumes may be dilations of lesion images.

It is thus a feature of at least one embodiment of the invention to provide a linkage system that is robust against minor mis-registration of the scans.

The output may characterize the lesions as appearing or disappearing and/or may indicate lesion volume or a change in various lesion measures between scans for individual or collections of lesions.

It is thus a feature of at least one embodiment of the invention to provide these useful measures in a system that more accurately links lesions between scans.

These particular objects and advantages may apply to only some embodiments falling within the claims and thus do not define the scope of the invention.

### BRIEF DESCRIPTION OF THE DRAWINGS

FIG. 1 is a simplified block diagram illustrating a set of medical images for processing according to the present invention on an electronic computer;

FIG. 2 is a flowchart showing the principal steps of the processing of the medical images of FIG. 1 to link lesions among different medical images to better assess lesion changes over time;

FIG. 3 is output display of lesion “temporal” tracks displaying histories of individual lesions as captured by the medical images at different points in time;

FIG. 4 is an alternative display superimposing the lesion tracks on the images;

FIG. 5 is a tabular output providing quantitative information about lesions categorized by organ according to the tracks established per FIG. 2;

FIG. 6 is a chart showing broad characterizations of lesions and possible mischaracterizations when the tracks are incorrectly established;

FIG. 7 is a plot over time of an individual lesion in the chart of FIG. 6 such as may be mischaracterized; and

FIG. 8 is a diagram indicating the relevant dimensions used in assessing whether a lesion has split or combined.

### DETAILED DESCRIPTION OF A PREFERRED EMBODIMENT

### System Hardware

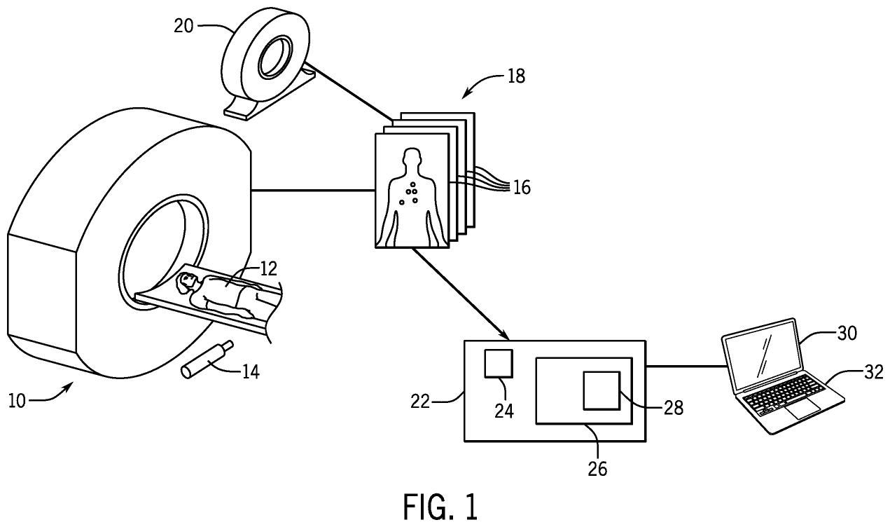

Referring now to FIG. 1, a scanner 10 capable of medical imaging from which one can identify lesions in a patient scan 12. The scanner 10, in one example, may be a PET (positron emission tomography) scanner producing three-dimensional image data (an image 16) revealing functional processes in the body reflected by migration of a molecular imaging agent 14 (e.g., a radioactive tracer) injected into the patient 12, preferentially to all or part of diseased tissue (henceforth “lesion”). The molecular imaging agent 14 in this case will be a positron emitting radio nucleotide attached to a biologically active molecule, the latter selected to participate in the lesion's molecular process.

Optionally, the functional images 16 may be supplemented or replaced with additional scans by other scanners 20, for example, a conventional kilovoltage or megavoltage CT (computed tomography), MRI (magnetic resonance imaging), or ultrasound system, such as may provide a higher resolution image 16 that presents anatomical information typically without the metabolic information. More generally, a given image 16 may be obtained from either scanner 10 or scanner 20 or may be a combination of data from multiple scanners 10 and 20 taken contemporaneously.

The images 16 present measures of multiple points in patient tissue each associated with volume elements (voxels) distributed in three dimensions, although only two dimensions are shown for clarity. The patient 12 will be imaged at different times during the course of a treatment of the patient 12 by chemotherapy, radiation therapy, or the like to provide a set of images 18 taken in sequence at different times.

The set of images 18 may be received by an electronic computer 22 for processing as will be described in greater detail below. Generally, the electronic computer 22 includes one or more processing units 24 communicating with a memory 26 holding data and a stored program 28 for effecting portions of the present invention. The computer 22 may communicate with a graphics display 30 for displaying color output images based on the images 16 and with user input devices 32 such as a keyboard, mouse, or the like, each allowing entry of data by user. The graphics display 30 will display an output indicating disease progression or regression based on measures of multiple lesion locations in the patient 12.

The invention will be described with respect to tracking metastatic lesions from cancer; however, the inventors contemplate that it may also be used with a variety of cancerous and noncancerous lesions including but not limited to skin lesions, retinal vascular network abnormalities, brain lesions related to Alzheimer's disease and multiple sclerosis, various polyps and cysts, arterial calcification, inflamed lymph nodes, etc.

### Program Operation

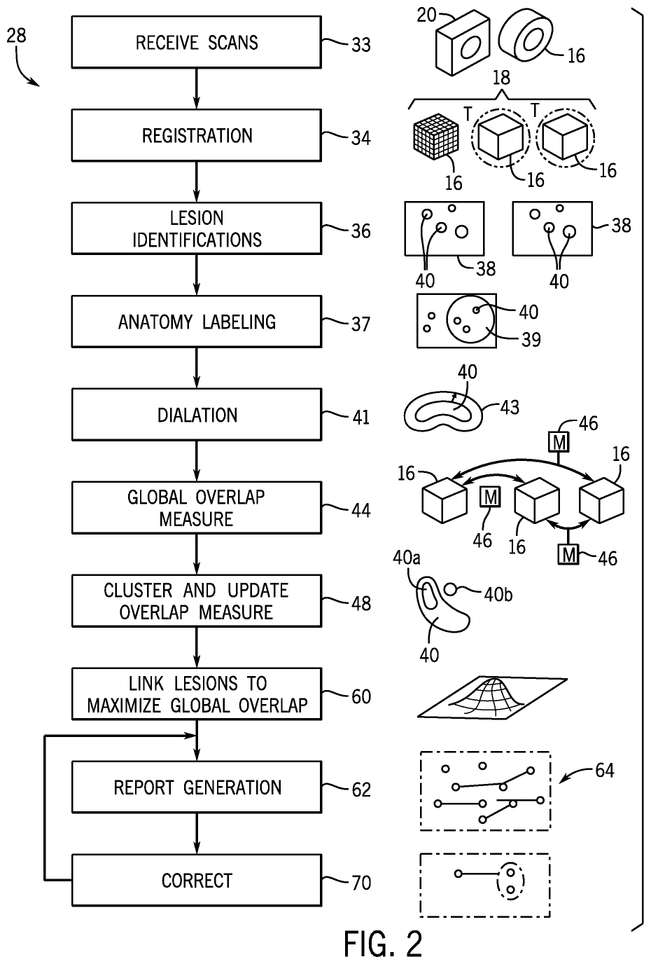

Referring now also to FIG. 2, the program 28 may accept, through input device 32, configuration information characterizing the type of cancer or other disease being treated, file names for accessing the images 16 of a set of images 18 (and hence image data of that set), and an identification of the type of imaging (for example, the type of molecular imaging agent being used), as indicated by process block 33. Nonlimiting examples of imaging with molecular imaging agents 14 include 18F-FLT (3′-deoxy-3′-[18F]fluoro-L-thymidine), a marker of cellular proliferation that quickly accumulates in proliferating cells that are synthesizing DNA and can be imaged with PET, and 18F-FDG (2-deoxy-2-[18F]fluoro-D-glucosc). 18F-FLT and 18F-FDG can be used to image upper tract urothelial, rectal, breast, lung (non-small cell), prostate, colon, ovarian, appendiceal, adenoid, and squamous cell cancers, and metastatic melanoma.

The invention further contemplates the use of other methods of distinguishing tissue lesions from healthy tissue such as parametric diffusion maps from diffusion-weighted magnetic resonance imaging (DW-MRI), single-photon emission computed tomography (SPECT) tracers, and even anatomical characterization without lesion-enhancing materials.

After acquisition, the images 16 may be registered or matched to each other using a three-dimensional registration process per process block 34. In one embodiment, all images 16 in the set of images 18 are registered to the baseline image, resulting in N−1 registrations of image pairs, where N is the number of images in a series. The intra-patient nature of the registration averts the need for registration atlases, which are commonly used for inter-patient registrations. For every image pair, the registration mode can be either direct or indirect; direct registration is defined between the baseline image and any other subsequent image, whereas indirect registration is defined as the registration of two subsequent images whose transformation fields were calculated to the baseline image. The registrations may be performed using a whole-body rigid registration (WRR) followed by whole-body deformable registration (WDR). In this case, the WRR performs an initial alignment of the images to prepare for the more detailed deformable step. In the WDR, a hierarchical control-grid B-spline free-form deformation (FFD) is used with a thin metal sheet bending energy regularizer per Rueckert, D., Sonoda, L. I., Hayes, C., Hill, D. L. G., Leach, M. O., & Hawkes, D. J. (1999), “Nonrigid registration using free-form deformations: application to breast MR images”, IEEE Transactions on Medical Imaging, 18(8), 18:712-21, hereby incorporated by reference. The optimization metrics may be the normalized mutual information for inter-modality registration (e.g., CT to MR) per Studholme, C., Hawkes, D. J., & Hill, D. L., (1998), “Normalized entropy measure for multimodality image alignment”, in K. M. Hanson (Ed.), Medical Imaging 1998: Image Processing (Vol. 3338, Issue June 1998, pp. 132-143), https://doi.org/10.1117/12.310835, hereby incorporated by reference. The normalized cross-correlation for intra-modality (e.g., CT to CT and MR to MR) when different modalities are used to collect the set of images 18 may be performed according to Avants, B. B., Epstein, C. L., Grossman, M., & Gec, J. C. (2008), Symmetric diffeomorphic image registration with cross-correlation: Evaluating automated labeling of elderly and neurodegenerative brain”, Medical Image Analysis, 12(1), 26-41. https://doi.org/10.1016/j.media.2007.06.004, also incorporated by reference.

Both metrics may be optimized using a gradient descent approach per Klein, S., Staring, M., Murphy, K., Viergever, M. A., & Pluim, J. (2010), “elastix: A Toolbox for Intensity-Based Medical Image Registration. IEEE Transactions on Medical Imaging, 29 (1), 196-205. https://doi.org/10.1109/TMI.2009.2035616, as incorporated by reference, with the cost function:

$$\hat{μ} = arg min [ C ( \vec{μ} ; {I}_{F} , {I}_{M} ) + ω R ( \vec{μ} ) ] \qquad ( 1 )$$

where C denotes the optimization metric (normalized mutual information or normalized cross-correlation) as a function of the moving image (I_{M}), the fixed image (I_{F}), and the set of parameters of the image transformation (\vec{μ}), which are translation and rotation parameters for WRR, and the parameters of a deformation field for the WDR per Sederberg. T. W., & Parry, S. R. (1986), “Free-form deformation of solid geometric models”, Proceedings of the 13th Annual Conference on Computer Graphics and Interactive Techniques, SIGGRAPH 1986, 20(4), 151-160. https://doi.org/10.1145/15922.15903, hereby incorporated by reference.

The function R(\vec{μ}) is a penalty term (bending energy) that enforces the smoothness of the transformation per S., L. L., & Wahba, G. (2006), “Spline Models for Observational Data. Mathematics of Computation”, 57(195), 444, https://doi.org/10.2307/2938687, hereby incorporated by reference.

Referring still to FIG. 2, at succeeding process block 36, a three-dimensional binary lesion mask 38 (Lₙ) may be prepared for each of images 16 representing a lesion 40, for example, at each voxel with a value of “1” and the absence of a lesion with a “0.” One method of generating the binary lesion mask 38 may evaluate the molecular imaging uptake values within the registered images 16 against a threshold, for example, of just above background uptake levels. Voxels of the patient 12 having the molecular imaging agent uptake values above the threshold may then be identified as lesions 40. Other imaging-identification techniques are contemplated including, for example, uptake gradient-based methods or machine learning methods. Within each image 16, the lesions are given an identification number 0−ω (where ω is the maximum number of lesions 40 in the image 16) that doesn't necessarily correspond to identification numbers of other images 16.

The optimal transformation field Tₙ (for image n) is then applied to the coordinates of the lesion masks Lₙ associated with each of the N images in the series, generating a registered lesion mask Lₙ_{_{T}}. The transformation field is the identity for n=1 (baseline image).

$${L}_{{n}_{T}} = {T}_{n} ( {L}_{n} ) \qquad ( 2 )$$

As indicated by process block 37, the lesions 40 of the binary lesion masks 38 may then be identified to a particular anatomical region 39 (body-part) where they are located. Example body parts include organs as well as regions such as the head and neck, chest (further detailing the lungs volume), pelvis, abdomen (further detailing the liver volume), spine, arms, and legs. This investigation may be performed by registering a whole-body segmentation atlas to the baseline image 16 of the set of images 18 and transforming it using the same registration procedure described above. Every lesion 40 is labeled according to the overlap of the lesion's volume to the body-part segmentation atlas. If more than one body-part 39 overlaps with a lesion volume, the lesion 40 is labeled according to the greatest volume of overlap.

Referring now to process block 41, the lesion contours of the lesion map are dilated to account for possible errors in the registration of the images 16 and increase the probability of lesion superposition. The dilation magnitude is decided independently for each lesion 40 and is based on the density of the lesion population in the anatomical region 39 where each lesion 40 is situated. A morphologic conformal lesion dilation operation is applied to each lesion in an image. The lesion-specific dilation magnitude (Dᵢ) is defined for each lesion as:

$${D}_{i} = min ( \frac{min ( {d}_{i , j} )}{2} , {D}_{m ax} ) \qquad ( 1 )$$

where d_{i,j} is the distance between a lesion i and every other lesion j in the same image, and Dₘₐₓ is a user-defined parameter to set a maximum allowed dilation magnitude. A new dilated lesion mask 43 (Lₙ_{_{T,D}}) is then defined as:

$${L}_{{n}_{T , D}} = {⋃}_{i} {D}_{i} ( {L}_{n , {i}_{T}} ) \qquad ( 2 )$$

where i indexes each lesion in the mask.

Referring still to FIG. 2, at succeeding process block 44, amounts of overlap 46 for each lesion 40 in each pair of images 16 are compiled. Specifically, the overlaps between the individual lesions (i and j) of different images 16 (n and q) are calculated for every possible image-pair in the series as:

$${M}_{n , {q}_{i , j}} = \vert {L}_{{n}_{T , {D}_{i}}} ⋂ {L}_{{q}_{T , {D}_{j}}} \vert \qquad ( 3 )$$

The matrices M_{n,q} are square matrices containing the volume of overlap between the lesions of time-points n and q. They have ω² elements where w is the maximal integer between the number of lesions identified in time-point n (ωₙ) and time point q (ω_{g}). The number of superposition matrices M_{n,q} created after the whole image series evaluation is N(N−1)/2. Note that this process compares not only sequential pairs of images 16, but, for example, also the first and last scan and all other combinations.

Referring to FIGS. 2 and 8, at succeeding process block 48, the possibility that lesions 40 have merged or split between images 16 is addressed through a clustering operation which links otherwise separate lesions 40 and 40′ in a single given image 16 as lesion 40. The clustering operation considers sequential pairs of images 16 in both scan order and reverse scan order according to a set of sub steps described in Table I below.

TABLE 1

| Sub-step | Description | Rationale |
|---|---|---|
| 1 | Determines a connecting orientation | The distances u and d will be |
| | \hat{t} between the lesion pair | measured along this orientation |
| 2 | Defines a characteristic separation | The distance u characterizes the |
| | distance u between both lesions in | separation of the lesion pair |
| | the pair, where u is aligned with | |
| | the orientation \hat{t} | |
| 3 | Projects the lesion pair contours | The measurement of d needs to |
| | along \hat{t} to constrain the common | be locally constrained (see d* |
| | intersecting lesion contour C | in FIG. 4D) |
| 4 | Determines the length d of the | The distance d defines a limit |
| | longest chord within the constrained | separation assuming the lesion |
| | common intersecting lesion contour | pair originated from lesion |
| | (C_{C}), where d is aligned with the | merging (or splitting) |
| | orientation \hat{t} | |
| 5 | Compares d and u to make the | Makes the final clustering decision |
| | clustering decision (positive for | |
| | cluster if d > u) | |

As shown in FIG. 8, a larger lesion 40 may be associated with two smaller lesions 40a and 40b in different sequential images 16 where the larger lesion 40 is in the first sequential image (in the case of lesion splitting) and the larger lesion 40 is in the second sequential image (in the case of lesion merging). For any two overlapping lesions (40 and 40a) all lesion pairs in a predefined neighborhood are considered for lesion merging.

In sub-step 1, the orientation \hat{t} is defined as the weighted mean of all the vectors 52 connecting each voxel of a given lesion (for example, lesion 40a) and adjacent lesions (for example, lesion 40b) within a predetermined distance 54 conservatively sized to capture any possible splitting or merging as follows:

$$\hat{t} = \frac{1}{V} {\sum }_{i = 1}^{N} {\vec{t}}_{i} \cdot {w}_{i} \qquad ( 4 )$$

where \vec{tᵢ} is a connecting vector, V is the total number of connecting vectors, and the weights wᵢ are defined as the inverse norm of each vector

$${w}_{i} = \frac{1}{\vert {\vec{t}}_{i} \vert} . \qquad ( 5 )$$

In sub-step 2, the distance u is determined as the 95^{th} percentile of the distribution of the norms of all vectors \vec{tᵢ} that satisfy the angular constraint:

$$u = {P}_{95} ( {\vec{t}}_{i} ) , subject to arccos ( \frac{{\vec{t}}_{i} \cdot \hat{t}}{\vert {\vec{t}}_{i} \vert \cdot \vert \hat{t} \vert} ) \leq 5 ° . \qquad ( 6 )$$

This imposes a soft constraint that the vectors being considered to determine u are aligned with \hat{t}. The 5-degrees value is a hyperparameter of the methodology that can be adapted according to each application. The 95^{th} percentile was chosen instead of a maximal operation because it is more robust against outliers in the distribution.

In sub-step 3, a constrained area of the lesion 40 (denoted by C_{c} and shown by hatching) is defined by the intersection lesion volume (C) of lesion 40 and an area defined by bounding lines (Lᵢ and Lⱼ) flanking the area of the lesions in the pair (40a and 40b) in the orientation \hat{t}, generating the projected areas (L_{i,p} and L_{j,p}). C_{c} is then defined as:

$${C}_{c} = {L}_{i , p} ⋂ {L}_{j , p} ⋂ C \qquad ( 7 )$$

In sub-step 4, \vec{d} is determined as the longest of the chords \vec{d}ᵢ within C_{c} subject to an angular constraint as:

$$d = max ( \vert {\vec{d}}_{i} \vert ) , subject to arccos ( \frac{{\vec{d}}_{i} \cdot \hat{t}}{\vert {\vec{d}}_{i} \vert \cdot \vert \hat{t} \vert} ) \leq 5 ° . \qquad ( 8 )$$

This enforces the chord \vec{d} being oriented in a similar orientation as \hat{t}, hence that the distances d and u are both measured along similar orientations.

Finally, in sub-step 5, u and d are compared, and if u<d the lesion pair 40a and 40b is considered as a clustered, rather than as two disconnected lesions 40.

Referring again to FIG. 2, as indicated by process blocks 60, the matching of lesion identifiers between images 16 can now be made by evaluating every possible image-pair combination in the series (N−1 combinations) to minimize a cost matrix K for each matrix M′_{n,q} as K_{n,q}_{_{i,j}}=1/M′_{n,q}_{_{i,j}}. Lesion matching is reduced then to a linear assignment problem of finding the optimal permutation matrices A_{n,q} that globally maximize the overlap between the lesions in the image-pair per Jaqaman, K., Loerke, D., Mettlen, M., Kuwata, H., Grinstein, S., Schmid, S. L., & Danuser, G. (2008), “Robust single-particle tracking in live-cell time-lapse sequences”, Nature Methods, 5(8), 695-702. https://doi.org/10.1038/nmeth.1237, hereby incorporated by reference:

$${A}_{n , q} = {arg min}_{{A}_{n , q}} ( {\sum }_{i = 1}^{ω} {\sum }_{j = 1}^{ω} {A}_{n , {q}_{i , j}} {K}_{n , {q}_{i , j}} ) \qquad ( 9 )$$

within the constraints to the permutation matrix A per:

$${\sum }_{i = 1}^{ω} {A}_{n , {q}_{i , j}} = 0 or 1 and {\sum }_{j = 1}^{ω} {A}_{n , {q}_{i , j}} = 0 or 1 \qquad ( 10 )$$

where the sum of the matrix elements can be zero if there are no matches for the lesions represented by those rows and columns.

The Munkres assignment algorithm per Munkres, J. (1957), “Algorithms for the assignment and transportation problems”, Journal of the society for industrial and applied mathematics, J Soc Indust Appl Math, 5(1), 32-38, as incorporated by reference, is used to find each optimal matrix A_{n,q} via a non-greedy minimization of the cost matrices K_{n,q}.

At process blocks 60, an uncertainty value may also be assigned to the selected matching. In this regard it should be noted that the superposition matrices M′_{n,q} contain the degree of overlap between the lesions in timepoints n and q. This matrix is used to calculate the cost matrix K_{n,q} with weights that are used as input to the linear assignment method of process block 60 to make the globally optimal lesion matching decisions. After the decisions have been made, the uncertainty (U) associated with each match (each graph edge) is calculated as the inverse of the intersection over minimum (IoM) associated with each match as follows:

$${U}_{n , {q}_{i , j}} = \frac{1}{{IoM}_{n , {q}_{i , j}}} = \frac{min ( \vert {L}_{{n}_{T , {D}_{i}}} \vert , \vert {L}_{{q}_{T , {D}_{j}}} \vert )}{\vert {L}_{{n}_{T , {D}_{i}}} ⋂ {L}_{{q}_{T , {D}_{j}}} \vert} = {K}_{n , {q}_{i , j}} \cdot min ( \vert {L}_{{n}_{T , {D}_{i}}} \vert , \vert {L}_{{q}_{T , {D}_{j}}} \vert ) \qquad ( 11 )$$

In this way, lesions with a high overlap have a low matching uncertainty and lesions with a low overlap have a high matching uncertainty, as demonstrated in FIG. 5.

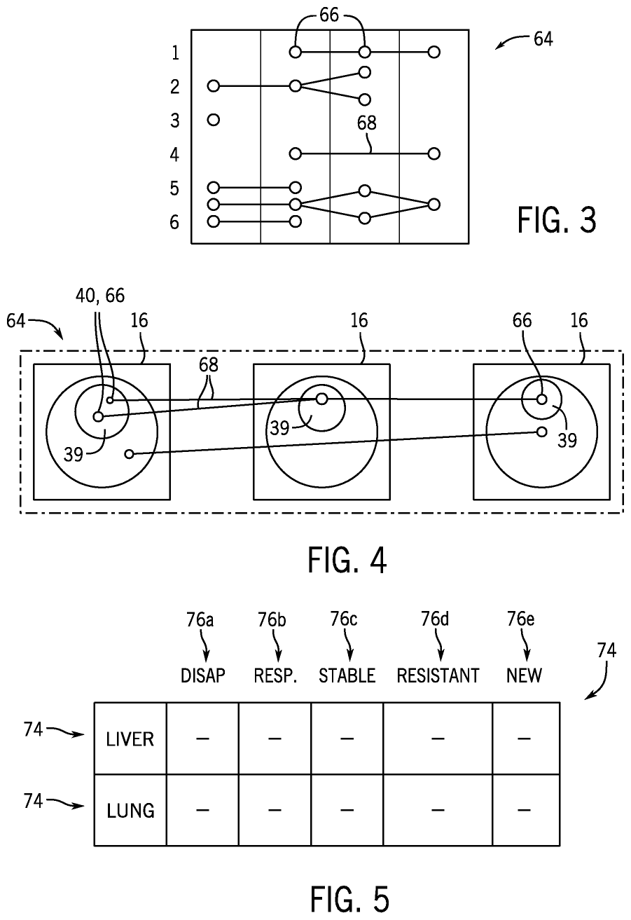

At this point, the lesion identifiers between images 16 have been linked so that lesion progression over multiple medical scans can be determined. Referring now to FIG. 3, this information may be used to generate a report indicated by process blocks 62, for example, summarized as a lesion graph 64 per Kuckertz, S., Klein, J., Engel, C., Geisler, B., KraB, S., & Heldmann, S. (2022), “Fully automated longitudinal tracking and in-depth analysis of the entire tumor burden: unlocking the complexity”, April, 86. https://doi.org/10.1117/12.2613080 and Yan, K., Wang, X., Lu, L., Zhang, L., Harrison, A. P., Bagheri, M., & Summers, R. M. (2018), “Deep Lesion Graphs in the Wild: Relationship Learning and Organization of Significant Radiology Image Findings in a Diverse Large-Scale Lesion Database”, Proceedings of the IEEE Computer Society Conference on Computer Vision and Pattern Recognition, 9261-9270.https://doi.org/10.1109/CVPR.2018.00965, both incorporated by reference.

This lesion graph 64 has one layer per image 16 in the set of images 18 (represented by a vertical column in FIG. 3) with nodes 66 representing lesions 40. Each individual node 66 in one layer represents one lesion in each time-point of a given image 16, if many lesion components are clustered, each component is still represented by an individual node 66 in the lesion graph. The edges linking the node 66 represent the temporal correspondence (temporal track 68) between the lesions 40 (i.e., the lesion matches determined globally at process block 60). A lesion temporal track 68 is defined by a connected subset of the graph (nodes at 66 and temporal tracks 68) that is disconnected from every other subset.

Referring now to FIG. 4, the lesion graph 64 may be alternatively displayed superimposed on the image 16 corresponding to the time point of each layer to provide improved context of the node temporal tracks 68. In this representation, the nodes 66 are sized and located in the images 16 according to their corresponding lesions 40. In addition, the individual depicted node 66 may be tagged with their uncertainty values as calculated above, for example, with numeric tags representing a degree of uncertainty or colors or shading or the like. Similar tagging may be done with the display of FIG. 3.

As shown in FIG. 2, using either of the displays of FIG. 3 or 4 and as indicated by process block 70, the user may modify the nodes 66 or the temporal tracks 68 by adding, removing, combining or separating nodes 66 based on the physician's knowledge and direct viewing of the images 16 to improve the resulting determination of temporal tracks 68. This process may be implemented, for example, by “clicking” on the node 66 to bring up a menu allowing splitting or joining or the other operations.

Referring to FIGS. 2, 4, and 5, the information obtained at process block 37 of FIG. 2 identifying organs or other tissue structures may be displayed on the images 16, for example, as outlines of the particular organs spatially located with respect to the respective lesions 40 or, as indicated in FIG. 5, as organ categories 74 in a table 73 providing statistics for the lesions 40 of those organ categories. Such statistics can include the number of lesions 40 that are disappearing per column 76a, the number of lesions 40 that are shrinking or responding per column 76b, the number of lesions 40 that are stable per 76c, the number of lesions 40 that are resistant to treatment and hence growing per column 76d, and the number of new lesions 40 per column 76c between any two designated images 16 or from the beginning or end of the set of images 18. In addition to number values, the total volume of the lesions may be indicated as well as a percent increase or decrease. This information may also be used to caption the displays of FIG. 3 or 4.

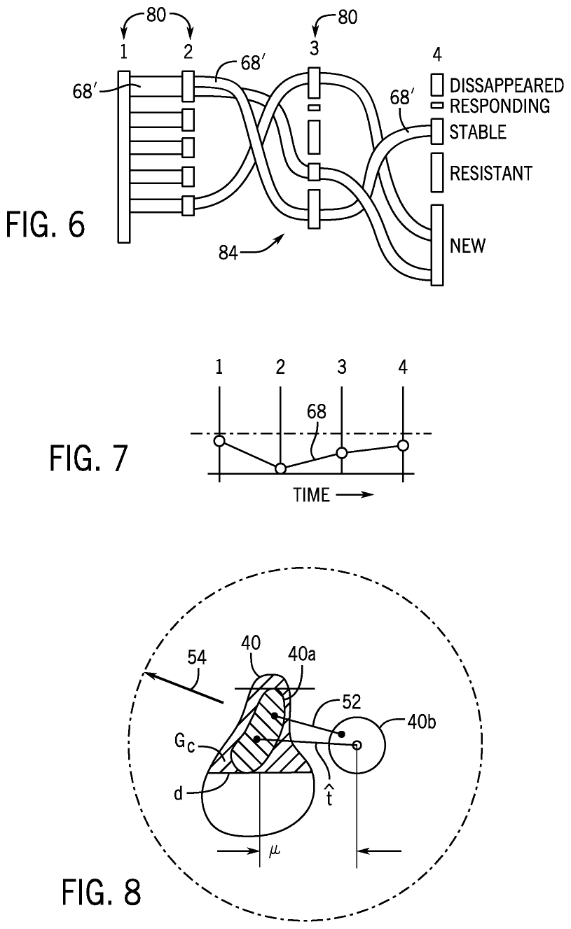

Referring now to FIGS. 6 and 7, the benefits of a global matching of lesions 40 as described above may be illustrated with respect to its advantage over a similar system that, for example, at given time intervals 80, corresponding to times of the acquisition of images 16, while performing only a linking of lesions by inspection of sequential pairs of images 16. In such a system, lesions 40 represented by a temporal track 68′ may, for example, disappear at a certain time interval 80 (2), for example, because of measurement error, being below a detectability limit, or short-lived remission, but ultimately resulting in a reduction in the lesion size. Without the global assessment of the present invention, such a temporal track 68′ would suggest incorrectly the appearance of a set of new lesions indicated by arrow 84 that eventually becomes stable when it is in fact this set of lesions that have been responding per track 68 of FIG. 7. The invention is also helpful in cases of lesion flare, where a lesion gets worse before getting better. More generally, the tracking of the present invention allows for a more accurate global assessment of lesion response, allowing for the capture of complex lesion evolution patterns.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to an electronic computer can be understood to include one or more computers that can communicate in a stand-alone and/or a distributed environment(s) or in the cloud, and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

What we claim is:

1. An apparatus for assessing treatment of a patient comprising:
an electronic computer executing a stored program to:
(a) receive a set of at least three scans of tissue of the patient revealing diseased tissue;
(b) determine lesion volumes in the scan as assigned to identifiers;
(c) determine an overlapping of lesion volumes between all pairs of scans of the set to provide a set of overlap measures for each pair of scans for each pair of identifiers;
(d) link pairs of the identifiers of different scans to globally maximize the overlap measures of the set over all of the scans; and
(e) output a display indicating a lesion change identified to given linked lesions.

2. The apparatus of claim 1 where in the electronic computer executing the stored program further identifies a set of tissue structures and wherein the output further identifies a lesion change to a tissue structure.

3. The apparatus of claim 2 wherein the tissue structures are organs.

4. The apparatus of claim 1 further including a graphic display and wherein the electronic computer executing the stored program further outputs a graphic display indicating a linkage between lesions of different scans superimposed on at least one scan image.

5. The apparatus of claim 4 wherein the electronic computer executing the stored program further: receives input from a user to alter the linking of (d) and after that alteration repeats (f).

6. The apparatus of claim 4 wherein the electronic computer executing the stored program further outputs uncertainty values on the graphics display associated with the linkage.

7. The apparatus of claim 1 wherein the lesion volumes are dilations of lesion images.

8. The apparatus of claim 1 where in the electronic computer executing the stored program further clusters lesions in a given image to present a combined lesion volume at (b).

9. The apparatus of claim 8 wherein the clustering is according to a distance derived from an overlapping lesion from an other scan of the pair.

10. The apparatus of claim 1 wherein the output characterizes the lesions as appearing or disappearing.

11. The apparatus of claim 1 wherein the output indicates lesion volume and change in other lesion measurements between scans.
